(19) United States
(12) Patent Application Publication
(10) Pub. No.: US 2026/0269088 A1
(43) Pub. Date: Sep. 10, 2026

(54) INFUSION DEVICE TO TRANSMIT NURSE CALL

(71) Applicant: CareFusion 303, Inc., San Diego, CA (US)

(72) Inventor: Michael K. WORKMAN, Carlsbad, CA (US)

(21) Appl. No.: 19/072,906

(22) Filed: Mar. 6, 2025

(51) Int. Cl. G16H 80/00; A61M 5/145; G16H 40/20

(52) U.S. Cl. CPC G16H 80/00; A61M 5/14546; G16H 40/20; A61M 2205/18; A61M 2205/3561; A61M 2205/505; A61M 2205/581

(57) ABSTRACT

Certain aspects of the disclosure provide a method of patient-nurse communication via an infusion pump system. In some examples, the method includes associating the infusion pump controller with the patient; receiving, on the infusion pump controller, user input corresponding to a nurse assistance call; and sending, to a nurse call system, information corresponding to the nurse assistance call.

### BACKGROUND

### Field

Aspects of the present disclosure relate to nurse call systems and methods, in particular, systems and methods for utilizing an infusion system to facilitate nurse calls.

### Introduction

Many hospitals and other patient care centers utilize nurse call systems to allow patients, family members, and other caregivers to remotely alert a nurse or other clinician to their need for help. For example, patients may use a button, cord, or other device to send a signal to a nursing station. Some systems will generate an alert or alarm at the nursing station to alert a nurse to the patient's need. The nurse would then need to visit the patient's room to determine the patient's need, and aid the patient. In some cases, the alert or alarm may be a light and/or sound on a nursing station. Some systems only allow the alert or alarm to be disengaged using the patient's button or cord, compelling the nurse to respond to the patient.

Other systems may facilitate a telephone call or intercom system to the nursing station, allowing a nurse to talk to the patient and determine the patient's need without leaving the nursing station. Then, the nurse may provide the assistance to the patient, in some cases, without leaving the nursing station.

Nurse call systems allow patients with limited mobility to contact the nurse because the call button, cord, or other device is often located nearby the patient, such as associated with a bed. For example, some patients may need assistance to get out of bed and may utilize the nurse call system to ask the nurse to help them get out of bed.

In some cases, a nurse call system may be utilized to alert a nurse to a patient emergency. For example, a patient may experience a medical emergency, and the patient may use the nurse call system to alert the nurse. Another person in the room with a patient experiencing a medical emergency may also use the nurse call system to alert the nurse, such as when the patient cannot do so themselves.

In other cases, a nurse call system may be utilized to alert a nurse to a non-urgent need or request, such as a family member desiring information about the patient's condition. In some cases, a nurse call system may be utilized to alert a nurse to an urgent, but non-emergent request, such as to assist the patient with getting to the bathroom or spill.

A conventional alert and alarm nurse call system, which only alerts at a nursing station to a request, uses the same alert for every call. For example, the emergency call, the urgent call, and the non-urgent call may all alert the same way at the nursing station. A nurse then needs to visit each patient to determine the nature of their call and provide the assistance. The nurse call system is unable to provide an indication as to a criticality or urgency of each call.

A nurse call system which enables telephone calls between a user and a nurse may help a nurse to understand the nature of each call because the nurse may talk to the user and gather more information than a simple alert or alarm. However, a nurse would need to remain at the nursing station to answer any incoming calls, otherwise a call may be missed.

Additionally, some patients and caregivers may overuse nurse call systems, for example, in non-urgent situations. Nurses may develop alarm fatigue due to frequent alarms and alerts. This may result in a missed call, or a delay in answering a call. Additionally, busy hospitals, multiple alerts or alarms may occur at the same time and nurses may need to attend to multiple calls at once. Further, excessive calls may interrupt nursing tasks, distract a nurse, or otherwise be disruptive to a nursing environment.

Accordingly, there is a need for improve nursing call systems and methods.

### SUMMARY

Certain aspects provide a method of contacting a nurse using an infusion pump system, the infusion pump system comprising an infusion pump coupled to an infusion pump controller configured to control the infusion pump to deliver fluid to a patient, the method comprising associating the infusion pump controller with the patient; receiving, on the infusion pump controller, user input corresponding to a nurse assistance call; and sending, to a nurse call system, information corresponding to the nurse assistance call.

Certain aspects provide a method of facilitating a nurse call, comprising receiving, from an infusion pump controller, information corresponding to a nurse assistance call; generating a response to the nurse assistance call; and sending, to the infusion pump controller, information corresponding to a response to the nurse assistance call.

Other aspects provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more aspects.

### DESCRIPTION OF THE DRAWINGS

The appended figures depict certain aspects and are therefore not to be considered limiting of the scope of this disclosure.

FIG. 1 depicts an example nurse call system.

FIG. 2 depicts an example workflow for activating a nurse call mode of an infusion system.

FIG. 3 depicts an example workflow for sending nurse call messages with an infusion system.

FIGS. 4A-4B depict example infusion system user interfaces for sending nurse call messages.

FIGS. 5A-5B depict additional example infusion system user interfaces for sending nurse call messages.

FIG. 6 depicts an example infusion system.

FIG. 7 depicts an example syringe infusion system.

FIG. 8 depicts an example method for contacting a nurse.

FIG. 9 depicts an example method for facilitating nurse-patient communications.

FIGS. 10-11 depict example computing devices with which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for sending nurse calls via an infusion system. In particular, systems and methods described herein provide for entry of nurse calls through the infusion system and infusion system-nurse call system connectivity. Nurse call messages may be related to the infusion system, or unrelated to the infusion system.

As described, nurse call systems are vital to providing care to patients, for example, those with limited mobility, those with emergent or urgent needs, or otherwise connect patients to nursing staff. Although described herein as a nursing call system, and connecting patients and nurses, a nurse call system may connect patients or caregivers to clinicians, including nurses, nursing assistants, physicians, or other providers.

Conventional nurse call systems utilize a button or cord located nearby a patient, or patient bed. For example, there may be a nurse call system button on a patient bed, on a controller for a patient bed, on a wall located nearby a patient bed, etc. In some cases, a pull-cord or button may be in places where patients frequent, such as a bathroom, exam room, or other care locations.

Such buttons or cords may alert or alarm at a nursing station, indicating where patient assistance is requested, such as an indicator of a patient room, a bathroom, an exam room, etc. However, conventional systems may not specifically alert the nurse as to which patient is requesting assistance, such as when more than one patient has access to a bathroom. Further, when a patient is away from their bed, such as in a bathroom, moving around a ward, visiting another care area, such as a testing center, the patient is also often away from the nurse call button or cord.

Further, as described, conventional nursing systems require a nurse to visit the alert location to respond to the call, or in the case of a telephone or intercom system, remain at the nursing station to receive the call.

Aspects of the present disclosure provide improved systems and methods for nurse-patient communications, by utilizing an infusion system as a nurse call messenger. Beneficially, infusion systems are often already conveniently located close to patients, and within patient care areas, such that a patient may generally be within reach of the infusion system, no matter where the patient is currently located. For example, as a patient travels to another care area, the bathroom, around a hospital room, the patient may remain with the infusion system because the infusion system is physically connected to the patient, e.g., through an intravenous line. Thus, by utilizing the infusion system as a nurse call messenger, patients, visitors, or caregivers may conveniently and efficiently contact a nurse.

Further, aspects described herein enable sending nurse call messages, containing additional information for a nurse, such as information about the nature of the request. Example nurse call systems described herein may utilize preset messages, such as common requests, or free-form messages, such as through text or voice entry, to provide information and context to a nurse call. Nurses then beneficially understand the nurse call, and the assistance requested before visiting the patient. For example, a patient may send a nurse call requesting an additional pillow. The nurse may obtain a pillow and bring it to the patient in one trip.

Additionally, information sent with the nurse call message may include a priority level, allowing a nurse to prioritize emergent or urgent calls over non-urgent calls. In some examples described herein, a priority level may be automatically assigned, such as through a machine learning model configured to assign a priority level based on the content of the message. Thereby, a priority level may be automatically assigned to calls.

Furthermore, in some cases, the nursing call system described herein may provide responses to nurse call messages at the infusion system. For example, a response may be displayed on a user interface of the infusion pump controller. The response may be an acknowledgement of the message, such as to confirm receipt. The response may be generated by a nurse, for example, with information responsive to the nurse call message. In some examples, the response may be generated automatically, e.g., based on the content of the message. A machine learning model, such as a language model, may be used to generate a response to the message, such as to provide patient information, hospital or care center information, instructions for use, or the like. Beneficially, non-urgent calls, or those that may be answered without nurse intervention may be responded to in a timely and efficient manner. Then, the volume of calls nurses respond to may be reduced and the number of trips to patient rooms may also be reduced. Beneficially, then, nurses may be disrupted fewer times, and interruptions may be for urgent and emergent calls, or those requiring nurse intervention to resolve.

Furthermore, aspects described herein provide technical benefits by utilizing preexisting network connections for providing secure transmission methods. Specifically, many infusion systems may be integrated within a hospital system to facilitate transmission of infusion data, such as infusion parameters, through secure network(s). Thus, nurse call messages sent through the infusion system may advantageously utilize these secure network(s) to transmit the entries, maintaining security of nurse-patient communications.

### Example Nurse Call System

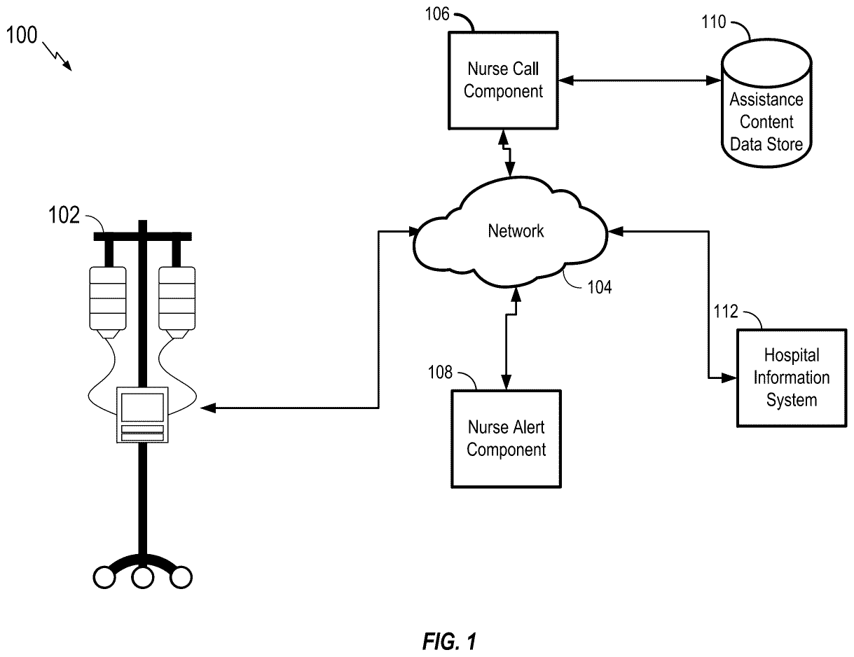

FIG. 1 depicts an example nurse call system 100 to facilitate usage of an infusion system for contacting a clinician, e.g., for calling a nurse or nurse's station.

Nurse call system 100 includes a hospital information system (HIS) 112 of the medical center. HIS 112 may include one or more computing devices, one or more databases, or the like. For example, HIS 112 is configured with an electronic medical recordation (EMR) system, a laboratory system, a pharmacy system, a medication ordering system, or the like, for the medical center, to facilitate communications such as transmitting and receiving laboratory testing data, prescription data, documentation of a patient's medical history, diagnosis, treatments, laboratory data, or the like.

Nurse call system 100 further includes an infusion system 102. Infusion system 102 is an electromechanical device for infusing therapeutic fluids into a patient in a controlled and precise manner. Exemplary infusion systems may include a syringe pump or a large volume pump. Infusion system 102 comprises an infusion system controller (not depicted) configured to control operations of infusion system 102. In general operation, therapeutic fluid is loaded into an infusion pump, for example, in a syringe, bottle, bag, or other storage container, and the infusion system is configured to pump, or infuse, the therapeutic fluid according to operational parameters of the pump, including, for example, flow rate, volume, and/or time to administer a specific dose of treatment.

Infusion system 102 is further configured to transmit data related to the infusion, for example, infusion parameters, infusion data, operational data, or the like. In some aspects, infusion system 102 is configured to transmit the data to HIS 112, for example, for entry into a subject's EMR.

As described herein, infusion system 102 is configured to send data to a nurse call component 106, which may then send a nurse call to a nurse alert component 108. In some aspects, data may be comprise a nurse call, e.g., a request for assistance, additional information, or the like, from a patient care team. In some aspects, the nurse call may be received via a user interface of the infusion system 102, e.g., an electronic display of the infusion system. For example, the user interface may comprise a one or more graphical control elements to facilitate entry of a nurse call. In another example, an infusion system may include one or more physical buttons (e.g., a softkey) to facilitate entry of a nurse call.

Although depicted here as separate from the infusion system 102, in some aspects, the nurse call component 106 may form part of the infusion system 102.

According to certain aspects described herein, infusion system 102 may receive data from nurse call component 106, for example, in response to the nurse call. In some aspects, infusion system 102 may display, on a user interface of infusion system 102, data received in response to the nurse call. For example, the infusion system 102 may display information answering the nurse call on the user interface of infusion system 102. In some aspects, the user interface may be on an infusion pump controller, adjacent to one or more infusion pumps, or otherwise associated with the infusion system coupled to the subject. Beneficially, a nurse call may be entered (e.g., by a subject or family member) via the user interface of infusion system 102, and a response received on the user interface, for display to the subject or family member.

In certain aspects, nurse call system 100 further includes a nurse call component 106 configured to receive nurse calls, process nurse calls, and/or transmit nurse calls to a nurse alert component 108. For example, nurse call component 106 is configured to receive data comprising a nurse call from the infusion system 102. In some aspects, nurse call system 100 may communicate with HIS 112, for example, to record data associated with a nurse call. In some aspects, nurse call component 106 is configured to transmit data associated with the nurse call to the nurse alert component 108. In some aspects, nurse call component 106 is configured to receive responses to the nurse call from the nurse alert component 108.

In some aspects, nurse call component 106 is configured to process nurse calls and generate a response. For example, in some aspects, a nurse call may comprise a preset request, and the nurse call component 106 is configured to send a preset response to the preset request to the infusion system 102 for display on the user interface. In another example, in some aspects, the nurse call component 106 may process the nurse call with a machine learning model, for example, a large language model (LLM), to generate a response. In yet another example, the nurse call component 106 may utilize an assistance content data store 110 to generate a response. In some examples, the nurse call component 106 may utilize content from HIS 112 to generate a response.

Nurse alert component 108 is configured to receive data associated with the nurse call. In some aspects, nurse alert component 108 is configured to display the data, such as on a user interface of a user device, a nursing station device, etc. For example, the data may be displayed as part of a notification of the nurse call. In some aspects, the nurse alert component 108 may receive data from a nurse, for example, a response to the nurse call via the user interface. The nurse alert component 108 may then transmit the response to the nurse call component 106.

Aspects of nurse call system 100 may communicate (e.g., send and/or receive), directly or indirectly via one or more devices using network 104 (e.g., a local area network (LAN), internet, etc.).

Although depicted as separate components, in some aspects, components of system 100 may be integrated, for example, nurse call component 106 may be integrated with the infusion system 102 or nurse alert component 108.

### Example Workflow for Nurse Calls via an Infusion System

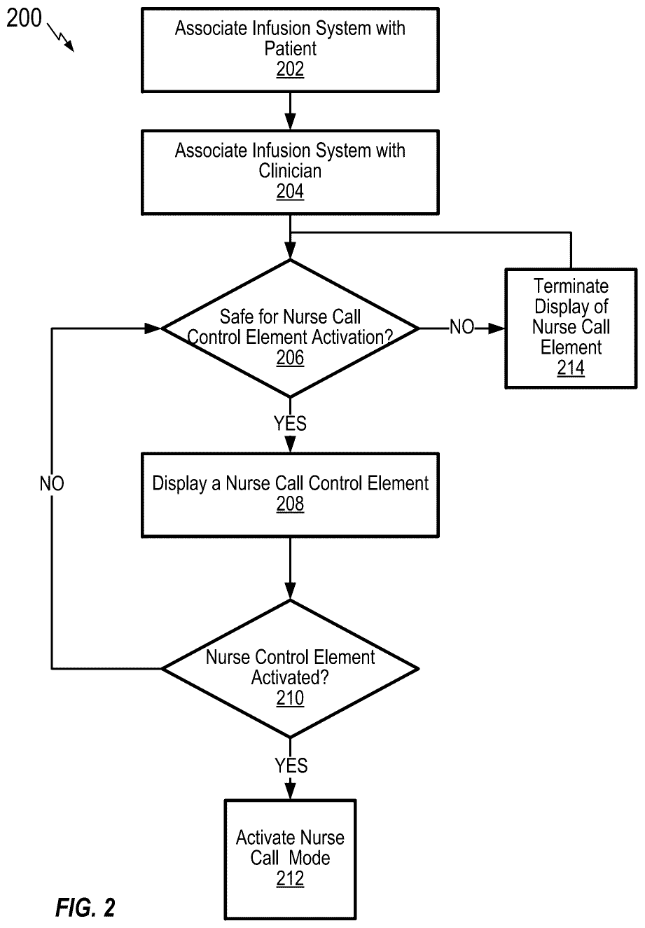

FIG. 2 depicts an example workflow 200 for entering a nurse call on an infusion system, such as infusion system 102 in FIG. 1.

Workflow 200 begins at step 202 with associating the infusion system, such as infusion system 102, with a patient. The infusion system may receive data associated with the patient, for example, a patient's name, a hospital identification number, etc., or another identifier of the patient. In some examples, a clinician may enter, such as through a user interface of the infusion system, the patient's identifier. In other examples, the patient identifier may be entered by scanning an identifier number, such as a barcode on a hospital bracelet, an electronic data tag, such as a radio frequency identification device (“RFID”) tag, or the like.

At step 204, optionally, the infusion system is associated with a clinician. The infusion system may receive data associated with a clinician, for example, a clinician's name, hospital identification number, etc. or another identifier of the clinician. In some examples, a clinician may enter, such as through a user interface of the infusion system, the clinician's identifier. In other examples, the clinician identifier may be entered by scanning an identification number, such as a barcode on an identification badge, an electronic data tag, such as an RFID tag, or the like. In some aspects, the infusion system is not associated with any clinician, for example, when a clinician identifier is not entered, a clinician identifier entered incorrectly, a clinician is not found, or the like, and the patient note mode is disabled until the infusion system is associated with a clinician.

At step 206, the infusion system determines whether it is safe for a nurse call control element to be activated. The infusion system includes an infusion pump controller configured to control operations of an infusion pump coupled to the patient. Because the infusion system may experience alarms or alerts related to operation of the infusion pump, for example, on the user interface of the infusion pump controller, the infusion system may prevent access to the nurse call mode during an active alarm or alert. Similarly, when the infusion system is in an unlocked mode, such as when a clinician is adjusting operational parameters of the infusion pump via the infusion pump controller, the infusion system may prevent access to the nurse call mode during operational adjustment. In some aspects, the infusion system may determine it is safe for a nurse call control element to be activated when the infusion pump controller is in a locked mode such that the operational parameters of the infusion pump may not be adjusted in the locked mode. Thus, a user may be restricted from adjustment of operational parameter(s). In some aspects, the infusion system may determine it is safe for a nurse call control element to be activated when the infusion system does not have an active alarm, for example, where the infusion system needs clinician intervention.

If at step 206, the infusion system determines it is not safe for the nurse call control element to be activated, then, at step 214, no nurse call element is activated, and display is terminated. The infusion system may return to step 206 and determine whether it is safe for the nurse call control element to be activated.

If at step 206, the infusion system determines it is safe for the nurse call control element to be activated, at step 208, the nurse call control element is displayed on the graphical user interface of the infusion system. The nurse call control element may be displayed on the graphical user interface associated with the infusion system. The nurse call control element controls activation of a nurse call mode allowing for communicating with a nurse or other clinician. Activation of the nurse call control element may include interacting with a portion of the graphical user interface (e.g., graphical control element) or pressing a physical button (e.g., a softkey) associated with the nurse call mode.

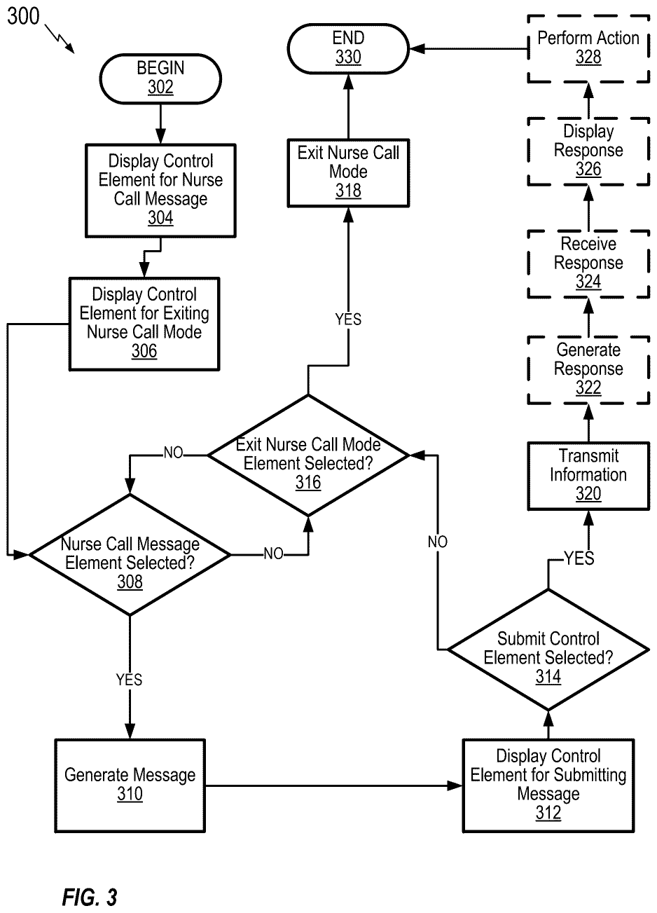

At step 210, the infusion system determines whether the nurse call control element is activated. The nurse call control element is activated when selected, for example, by a patient or other using a button on the infusion system, selecting on the user interface of the infusion system, issuing a voice command (e.g., “Infusion Helper, please call the nurse”), or the like. At step 212, the nurse call mode is activated on the infusion system. Where a nurse call mode is activated on the infusion system, for example through workflow 200, or another suitable workflow, the infusion system may utilize workflow 300 to facilitate entry of the nurse call. In particular, FIG. 3 depicts an example workflow 300 for calling a nurse (or other clinician) through a nurse call mode.

Workflow 300 begins at step 302 during a nurse call mode, for example, following activation of a nurse call mode at step 212 in FIG. 2.

At step 304, a control element for a nurse call message is displayed on the user interface of the infusion system, for example, a graphical user interface of the infusion system controller. In some aspects, a nurse call message control element may be for a preset nurse call message (also called quick call or quick message). In some aspects, one or more preset nurse call messages may be displayed, for example, as individual control elements on the user interface.

Exemplary nurse call messages may include: “Patient needs assistance;” “Patient environment needs assistance” or “Patient medical device needs assistance.” Preset nurse call messages may be defined by a manufacturer of the infusion system, for example, pre-programed on the infusion system. Preset nurse call messages may be defined by the hospital system and transmitted, for example, through the network, to the infusion system. Preset nurse call messages may be based on one or more of: a location of the infusion pump, a clinician type, a patient type, one or more infusion parameters, or the like. For example, the one or more preset nurse call messages for an infusion system in a neonatal intensive care unit may be different from the one or more preset nurse call messages for an infusion system in an adult intensive care unit. The preset nurse call messages may then be beneficially relevant to the patient, clinician, or location of the infusion system.

In some examples, a preset nurse call message may be a message to initiate a telephone call with the nurse, for example, through the nurse alert component 108.

In some aspects, a nurse call message control element may be for free-form nurse call message entry, for example, a text entry, a speech-to-text entry, a voice recording entry, etc. For example, free-form text entry message is generated by a user inputting text using a keyboard, for example, a keyboard on a user interface, or one or more buttons on the infusion system. In some aspects, a free-form text entry message is generated based on receiving speech, for example, using a microphone associated with the infusion system. The infusion system may utilize a speech-to-text model to generate a text entry comprising the user's speech. In some aspects, a free-form nurse call message entry may be a recording of the user's speech, e.g., through a microphone associated with the infusion system.

In some examples, speech recognition may be used to transcribe speech into text for the message. For example, natural language processing (NLP) techniques may be used to process and transcribe the recording of the user's speech into text. In some examples, a machine learning model may be used for speech transcription, such as a language model. A language model is a sophisticated NLP tool that analyzes and generates human language by understanding the probabilistic relationships between words and leverages large datasets to learn these relationships. Language models are often trained on large corpora of text. The training process involves adjusting the model's parameters to minimize the different between its predicted word probabilities and the actual word sequences in the training data. Such training may be done using techniques like maximum likelihood estimation and gradient descent.

Though certain aspects are discussed with respect to an infusion system allowing both preset nurse calls and “free form” nurse call messages that are not preset, other workflows may similarly be used that support only preset nurse call messages, or only text nurse calls that are not preset.

At step 306, a control element for exiting the nurse call mode is displayed on the user interface of the infusion system, for example, a graphical user interface of the infusion system controller.

At step, 308, it is determined whether the control element associated with the nurse call message is selected, for example, by a user using the user interface of the infusion system, such as on the user interface, or a softkey associated with the control element on the user interface. If the nurse call message control element is not selected at step 308, then workflow 300 proceeds to step 316.

If, at step 308, the nurse call message control element is selected, then workflow 300 proceeds to step 310 with generating a message based on the selection. For example, where the nurse call message control element selected is associated with a preset nurse call message, the preset nurse call message is generated. As another example, where the nurse call control element selected is a free-form text entry, a message is generated based on a user inputting text using a keyboard, such as with a keyboard on a user interface, or one or more buttons on the infusion system. In another example, when the nurse call control element selected is a free form speech entry, a text message is generated based on processing the user's speech to generate a text message. In yet another example, when the nurse call control element selected is a voice recording entry, a voice recording is generated based on recording the user's speech. As another example, when the nurse call control element selected is a telephone call with the nurse, information to establish a telephone call is used to generate the message.

At step 312, a control element for submitting the message is displayed on the user interface, whether the message is generated based on a preset message or a free-form message.

Workflow 300 proceeds to step 314 with determining whether the control element for submitting the message is selected. If, at step 314, the control element for submitting the message is not selected, then workflow 300 proceeds to step 316 with determining whether the control element for exiting the nurse call mode is selected. If, at step 316, the control element for existing the nurse call mode is not selected, workflow 300 returns to step 308. If the control element for exiting the nurse call mode is selected, then workflow 300 proceeds to step 318 with exiting the nurse call mode. In some cases, after exiting the nurse call mode, workflow 300 may return to step 206 of workflow 200, with determining whether it is safe for activation of a nurse call mode.

If, at step 314, the control element for submitting the message is selected, then workflow 300 proceeds to step 320 with transmitting information comprising the message, for example, using the nurse call component 106 to transmit the information to the nurse alert component 108. The nurse alert component 108 may be associated with a nursing device, such as a mobile device of the nurse, and the message may be transmitted for display on the nursing device. In some aspects, the information may be routed to nursing device based on the clinician associated with the infusion system.

In some aspects, the nurse alert component 108 may be associated with a nursing station device, for example, a nurse monitoring device at a nurse station, or other clinician station, and the information may be transmitted for display on the nursing station device. For example, in some aspects, such as where the infusion system is not associated with a clinician (e.g., workflow 200 skips step 204), the message may be transmitted to a nursing station device. In some aspects, the nursing station device may be identified based on patient information, for example, a location of the patient (e.g., a care area, a room or bed number, etc.), and the nursing station device may be determined as the nursing station device for that location.

In some aspects, the information is further transmitted to a patient's EMR for documentation. The information may be formatted to conform with a standardized electronic medical messaging format such as HL7 Clinical Note or similar.

The information includes the message generated at step 310, for example, based on the preset nurse call message, the free-form entry, the speech-to-text entry, or the voice recording. In some aspects, the information further includes an identifier of the patient currently associated with the infusion system. In some embodiments, the information additionally or alternatively comprises an identifier of the clinician currently associated with the infusion system. The inclusion of the clinician identifier or patient identifier may be performed automatically (e.g., without user input during the message entry), relying on the current association between the infusion system, patient, and clinician (e.g., an association established during prior initialization of the infusion system). In some aspects, the information additionally or alternatively comprises at least one of: a time stamp of when the user input was received (e.g., selection of a preset nurse control element or free-form text entry received), an identifier associated with the infusion pump controller, or an identifier of a location of the infusion pump controller. The information may be formatted according to a proprietary standard such as a well-defined XML or JSON format.

In some aspects, the information may include a priority level associated with message. For example, the priority level may be associated with a preset nurse call message and determined based on selection of the preset nurse call message. In another example, the priority level may be selected by a user as part of inputting the message, for example, with selection of the preset nurse call message or free-form text entry.

In some aspects, the priority level may be determined based on the content of the message. For example, the priority level may be determined based on a sentiment of the content of the message. Sentiment analysis is an NLP technique to determine sentiment expressed in a piece of text or audio. A priority level may be higher where the sentiment is determined to be negative or has high intensity.

In some aspects, the priority level may be determined based on the voice recording, for example, background noise. Analysis of background noise may indicate an alarm or alert in in the environment of the infusion system, or other activity in the environment of the infusion system. For example, where an alarm or alert is detected in the background, a higher priority level may be determined.

Optionally workflow 300 proceeds to step 322 with generating a response to the message. In some aspects, generating a response to the message comprises generating an acknowledgment of receipt of the message by the nurse alert component 108. In some aspects, the response is further transmitted to a patient's EMR for documentation.

In some aspects, generating a response to the message comprises receiving, from the nurse (or other clinician), the response, for example, by entry through the nurse alert component 108. A nurse may generate a text response or a voice response to the message. In some examples, the nurse alert component 108 may provide (e.g., suggest) one or more preset responses to the preset nurse call message for selection by the nurse. In other examples, the nurse alert component 108 may facilitate entry of a free-from entry, such as a text or voice entry.

In some aspects, such as where the message comprises a request for a telephone call, the response may comprise information for establishing a telephone connection with the infusion system (e.g., the user of the infusion system and the nurse). For example, a voice call may be established between the user and the nurse.

In some aspects, the response comprises a self-help message responsive to the message. A self-help message, for example, may provide information related to the user's request, such as information about the patient or guidance for the user to resolve their message.

In some aspects, generating a response to the message comprises generating a human-understandable response through natural language generation (NLG). For example, a language model may generate text responsive to the message. Example language models include those based on transformer architectures, which use an encoder-decoder structure and do not rely on recurrence and/or convolutions to generate an output. Exemplary transformer-based models include bi-directional encoder representations from transformers (BERT) and generative pre-trained transformers (GPT). Other language models may include long short-term memory (LSTM) models which are based on recurrent neural network architectures.

A language model may process a text prompt comprising the message and generate a text response. In some cases, a language model may use additional information to generate the text response (e.g., in addition to knowledge gained through training). For example, additional information may include patient-specific information. In some cases, additional information may be obtained from the HIS or the patient's EMR. Such additional information may be provided to the model with the message as part of the prompt.

In some cases, the language model may utilize retrieval augmented generation (RAG) to supplement knowledge gained through training. In models configured with RAG techniques, the model uses a data retrieval component to select relevant information from external content data storage, such as data store of the HIS or EMR. The model then uses the relevant information along with the prompt to generate the response.

For example, a nurse call message may comprise a request for information about an upcoming procedure for the patient. The language model may use RAG to identify and select information from the HIS and the patient's EMR, such as a scheduled endoscopy for the patient, and information about various procedures. The language model may use the patient's request and the additional information to generate a response including the endoscopy scheduled for the patient and information about endoscopies.

Optionally, workflow 300 proceeds to step 324 with receiving the response to the message at the infusion system. The response may be received from the nurse alert component 108.

Workflow 300 then proceeds with step 326 with displaying the response on the user interface of the infusion system. For example, the response may be displayed as a pop-up on the user interface of the infusion system. In some aspects, the infusion system may be configured to use text-to-speech to generate an audio read out of the response and use a speaker associated with the infusion system to play the audio read out of the response. In some cases, the audio read out may be play simultaneously with the displaying of the response.

In some aspects, the response may comprise a voice response, for example, recorded by a nurse using the nurse alert component 108, and a speaker associated with the infusion system may play the voice response.

Optionally, workflow 300 may proceed with step 328 by performing an action at the infusion system. In some cases, the action may be determined based on the nurse call message. In other cases, the action may be determined based on the response to the nurse call message. In some aspects, the response may comprise an action for the infusion system, for example, an adjustment to one or more operational parameters of the infusion pump. An action may include silencing an alarm, pausing an infusion operation, starting, or restarting the infusion operation, rebooting an infusion pump, or the like. For example, as part of a nurse entered response, an adjustment to an operational parameter of the infusion pump may be entered. The adjustment to the operational parameter may be implemented by the infusion system upon receipt of the response (e.g., at step 324). Workflow 300 then ends at step 330.

Note that workflows 200 and 300 are just examples, and other flows including fewer, additional, or alternative steps, consistent with this disclosure, are possible. As discussed, a control element described in FIG. 3 may include a physical button or an element shown on a graphical user interface that can be activated or mapped to a physical button (e.g., a softkey). Activation of the control element may include interacting with a portion of the graphical user interface (e.g., button) or pressing a physical button (e.g., a softkey) to cause the transmission of a message indicating the interaction with the control element.

### Example Infusion System User Interfaces

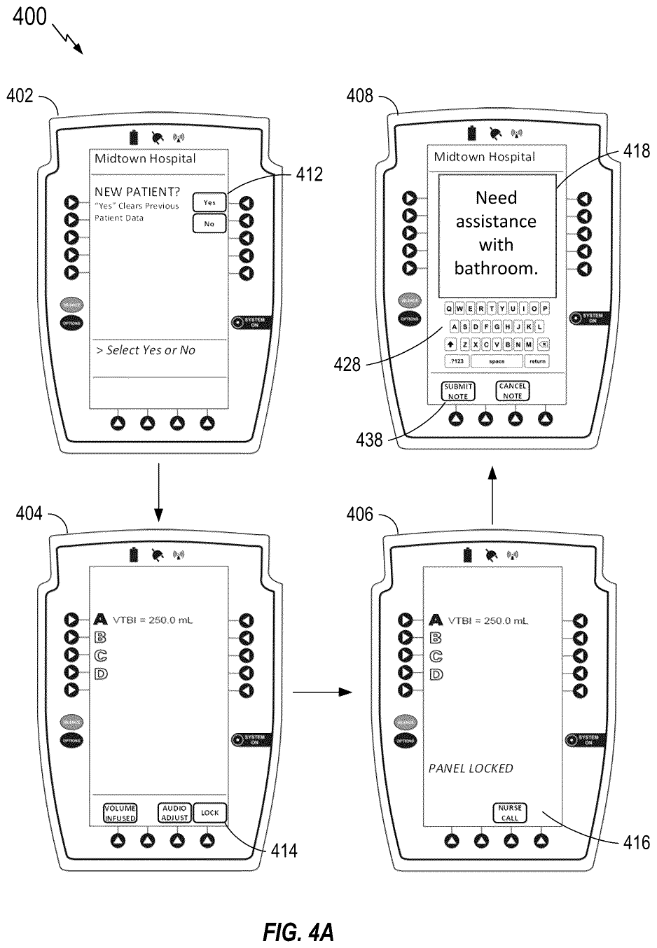

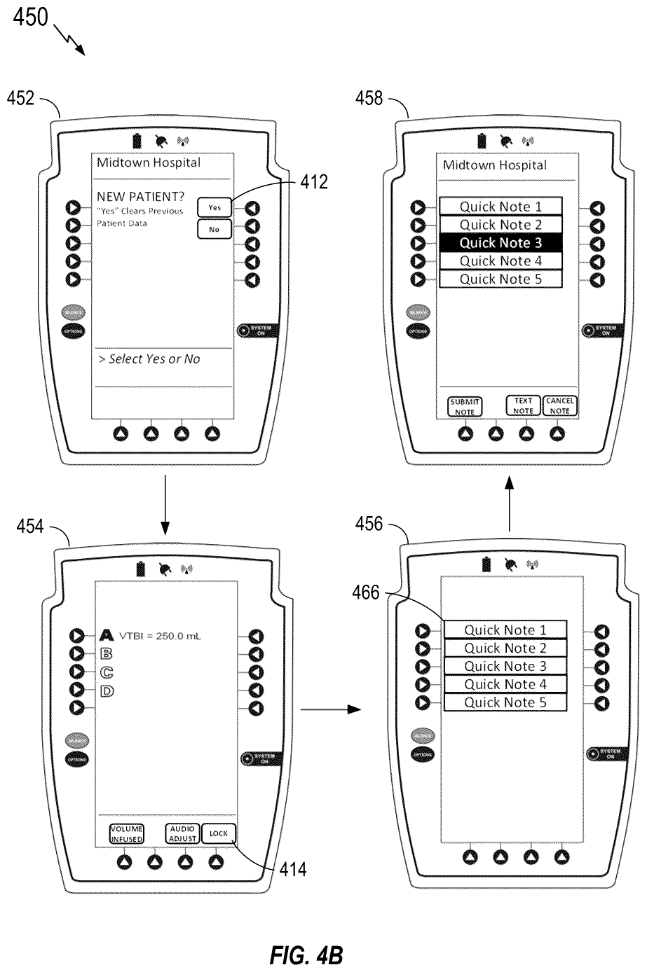

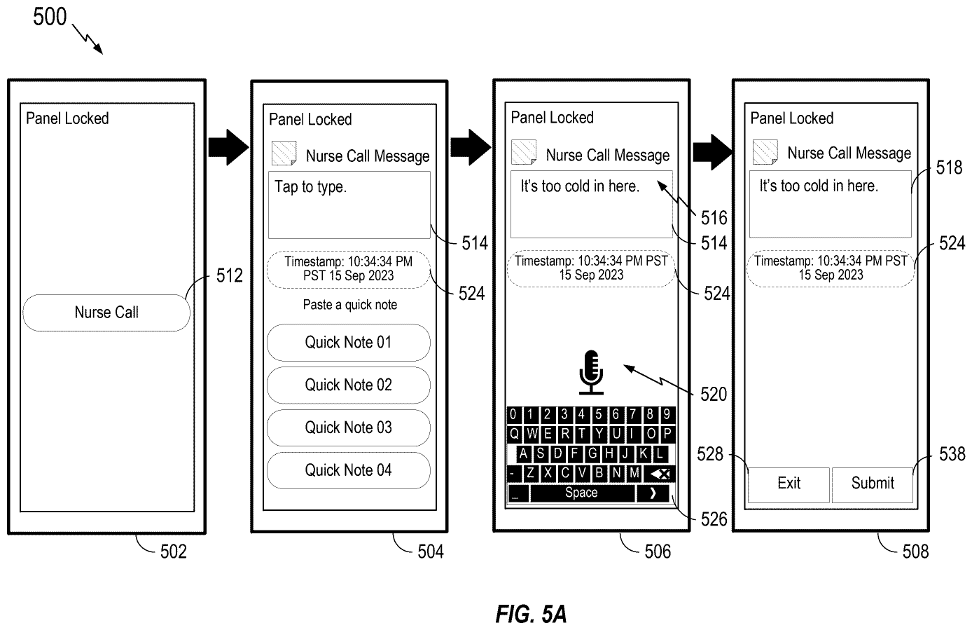

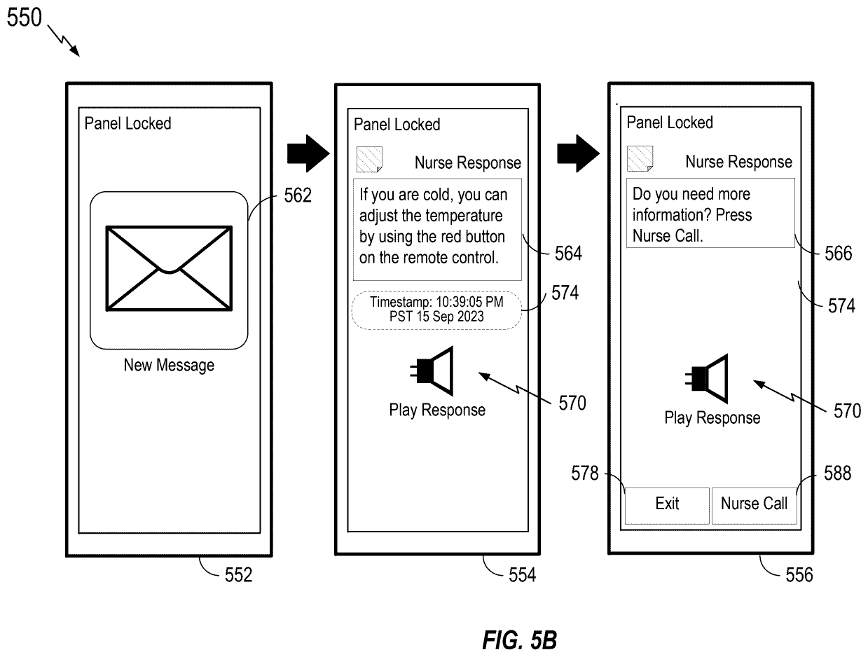

FIGS. 4A-4B and 5A-5B depict example user interfaces of an infusion system performing aspects of workflows 200 and 300, described with respect to FIGS. 2 and 3. In particular, FIG. 4A depicts an example flow 400 of an infusion system user interface for sending nurse call messages via free-form text entry.

Initially, the infusion system user interface 402 displays a patient association control element 412, such as to associate a patient with the infusion system, as described with respect to step 202 of FIG. 2. A clinician may interact with the infusion system user interface, for example through one or more buttons, on-screen selections, or the like, to enter patient details. Note that prior to associating with a patient, the infusion system user interface 402 does not include a nurse call control element.

In some aspects, the infusion system user interface further displays a clinician association control element (not shown), to associate a clinician with the infusion system, such as described with respect to step 204 of FIG. 2. A clinician may interact with the infusion system user interface to associate the infusion system with the clinician.

In some aspects, the clinician further uses the infusion system user interface to operate the infusion system, for example, set up an infusion for the patient, adjust one or more infusion operation parameters, or the like.

In some aspects, after the clinician is finished interacting with the infusion system, the clinician may utilize a lock control element 414 on user interface 404 to lock the infusion system.

Once the infusion system is locked, the infusion system user interface 406 displays a nurse call mode control element 416, such as to activate a nurse call mode, as described with respect to step 212 of FIG. 2. For example, a patient may utilize the nurse call mode control element 416 to call a nurse. In another example, another user, such as a family member or other caregiver may utilize the nurse call mode control element 416 to call a nurse.

In the nurse call mode, the infusion system user interface 408 displays an element 418 for free-form text entry, such as to facilitate reception of the free-form text entry message, as described with respect to step 308 of FIG. 3. In the depicted example, the free-form text entry includes “Need assistance with bathroom.”

Although depicted here as a QWERTY keyboard 428, other keyboards may be used to facilitate efficient text entry such as a telephone keypad, such as for T9 input. Further, a speech-to-text control element may be configured to receive speech, such as through a microphone, and convert the speech to a free-form text entry. Other suitable methods of entering free-form text may also be used.

Additionally, in some aspects, a voice recording element may be displayed and configured to record a voice recording (e.g., a voice note or a voice memo).

The nurse call message may be submitted, for example, by selection of submit message element 438. Submitting the message may transmit a formatted message including the message, patient identifier, and clinician identifier, from the infusion system via a nurse call component to the nurse or nursing station via a nurse alert component. The transmission may be direct or via an intermediate system such as via an infusion system communication gateway. After submission of the message, the infusion system may exit the nurse call mode and return to the locked state, such as depicted in user interface 404, such as described to step 206 in FIG. 2

FIG. 4B depicts an example flow 450 of the infusion system user interface for sending nurse call messages via one or more preset nurse call messages.

User interface 452 and user interface 454 may operate as described with respect to user interfaces 402 and 404 described in FIG. 4A.

However, in flow 450, in the nurse call mode, the infusion system user interface 456 displays a preset element 466 comprising one or more preset nurse call messages, depicted here as Quick Notes 1-5. As described with respect to steps 308 of FIG. 3, the preset element 466 may be displayed where one or more preset nurse call messages are defined and then may be displayed. Beneficially, these one or more preset nurse call messages may be commonly used messages or user needs. For example, preset nurse call messages may relate to patient needs, such as assistance with moving about the patient's environment, questions regarding care, additional concerns, food and/or drink needs, or the like. Preset nurse call messages may relate to environmental needs, such as assistance with changing linens, collecting trash, cleaning, or the like. Preset nurse call messages may be related to medication and/or medical devices, for example, assistance with a medical device coupled to the patient, another medical device, questions about medication or treatment, or the like. Beneficially, preset nurse call messages may be utilized by various users, for example, a patient, a caregiver, or a family member, to obtain assistance for the patient.

The infusion system user interface 458 displays a selection of Quick Note 3 for submission of the nurse call message.

FIG. 5A depicts another exemplary flow 500 of the infusion system user interface for sending nurse call messages; beneficially, flow 500 depicts aspects of both free-form text and preset entry.

User interface 502 displays multiple control elements, including a nurse call control element 512. After selection of the nurse call control element 512, nurse call mode is activated, for example as described at step 212 of FIG. 2.

User interface 504 displays a text note control element 514, as well as control elements associated with Quick Notes 01-04. In the depicted example, a timestamp 524 is also depicted and may be associated with the entry.

User interface 506 displays a free-form text entry 516, as well as a QWERTY keyboard 526 to facilitate entry. User interface 506 also displays a voice recording element 520 to facilitate recording of a message or speech-to-text entry.

User interface 508 depicts the nurse call message 518, the optional associated timestamp 524, and control elements for exiting the nurse call mode 528 or submitting the nurse call message 538. When the nurse call message submission control element 538 is selected, the nurse call message is generated and sent to the nurse, such as described with respect to step 320 in FIG. 3. When the exit control element 528 is selected, the user interface exits the nurse call mode, such as described with respect to step 318 in FIG. 3.

FIG. 5B depicts an example flow 550 of the infusion system user interface for receiving responses to a nurse call message, such as the nurse call message 518.

User interface 552 displays an alert of a new message 562, received in response to a previous nurse call message. In some cases, the new message alert 562 may also include an audible alert.

User interface 554 displays the text of the response 564, in this example, instructions for adjusting a temperature. As described herein, in some examples, the response may be a response entered by a nurse based on the nurse call message. In other examples, the response may be generated by the nurse call system in response to the nurse call message, for example, a self-help message. In some cases, the response may comprise an acknowledgment of the receipt of the nurse call message by the nurse call system.

In the depicted example, the user interface 554 also displays a timestamp 574 associated the response 564.

User interface 554 also displays an audio control element 570 configured to read out, e.g., using text-to-speech, the response 564.

User interface 556 displays a second response 566, asking the user to confirm the response resolved their concern. A nurse call control element 588 enables sending another nurse call message, e.g., returning to flow 500. User interface 556 also displays an exit control element 578, which, when selected, the user interface exits the nurse call mode, such as described with respect to step 318 in FIG. 3.

### Example Infusion System

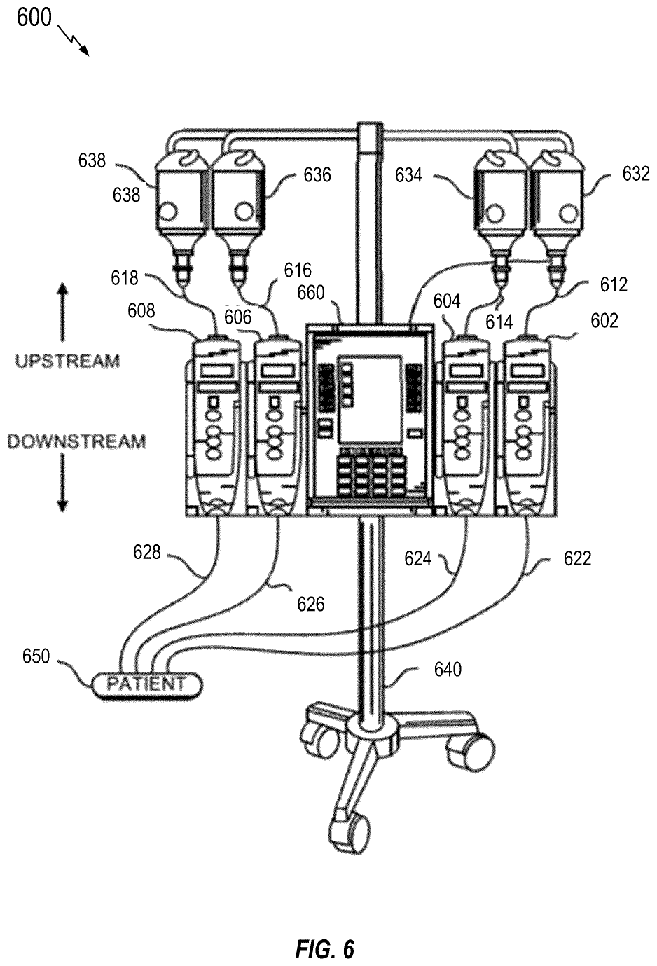

FIG. 6 depicts an example infusion system 600, which may implement methods described herein for sending and receiving nurse call messages. The infusion system 600 may be coupled to a patient 650 to administer a substance to the patient 650 through infusion. The infusion system 600 may include one or more pumps, for example, pump 602, pump 604, pump 606, and pump 608. Although a large volume pump is illustrated, other types of pumps may be implemented, such as a peristaltic pump, a small volume pump, a syringe pump, an anesthesia delivery pump, or a patient-controlled analgesic. A pump may be an infusion device configured to deliver a substance, (e.g., fluid, nutrients, drug, etc.) to a patient's circulatory system, or epidural space, for example, via an intravenous infusion, subcutaneous infusion, arterial infusion, epidural infusion, etc., or to a patient's digestive system, for example, via a nasogastric tube (NG), a percutaneous endoscopic gastrostomy tube (PEG), nasojejunal tube (NJ), etc. Each of the pumps 602, 604, 606, or 608 may comprise a standard infusion pumping unit, patient-controlled analgesia (PCA) pump, syringe pump, pulse oximeter, invasive or non-invasive blood pressure monitor, electrocardiograph, bar code reader, printer, temperature monitor, RF telemetry link, fluid warmer/IV pump, or high rate IV pump (2000+ ml/hr).

Each of the pumps 602, 604, 606, or 608 may be fluidly connected with an upstream fluid line 612, fluid line 614, fluid line 616, and fluid line 618, respectively. Further, each of pump 602, pump 604, pump 606, and pump 608, may be fluidly connected with a downstream fluid line 622, fluid line 624, fluid line 626, and fluid line 628, respectively. The fluid lines may be any type of fluid conduit, such as tubing, through which fluid can flow.

Each of fluid supply 632, fluid supply 634, fluid supply 636, and fluid supply 638, may be a reservoir, for example, as bottles shown, inverted, and suspended above the pumps. Fluid supplies may also take the form of bags, syringes, or other types of containers. The medical device system 600 may be mounted on a roller stand or intravenous pole 640.

Infusion pump system 600 may further comprise check valves, drip chambers, valved ports, connectors, and other devices configured to administer a substance.

Each of the pumps 602, 604, 606, or 608 of infusion pump system 600 may be coupled to an infusion pump controller 660. The infusion pump controller 660 may be a patient care device (PCD) or patient care unit (PCU). The infusion pump controller 660 is configured to control operations of the pumps 602, 604, 606, or 608, for example, to control infusions to patient 650. The infusion pump controller 660 may be further configured to the control of fluid delivery to the patient and the monitoring of the fluid path for occlusion or air-in-line obstructions.

The infusion pump controller 660 may further include a graphical user interface (GUI), for example, an information display such as a liquid crystal display, and/or a touch-screen. The user interface of the infusion pump controller 660 may be used during set up and/or operating procedures to facilitate control of the infusion system 600. For example, entry, editing, and/or display of operational parameters of the pumps 602, 604, 606, or 608. The infusion pump controller 660 may further include one or more softkeys and/or hardkeys to facilitate data entry and commands. In some aspects, a user interface may display the operational parameters of the pumps 602, 604, 606, or 608, for example, the infusion rate at which the pump is operating. The user interface may additionally and/or alternatively, be used to display informational, advisory, alarm, or malfunction messages associated with the pumps 602, 604, 606, or 608.

The infusion pump controller 660 may further be configured to provide power to the infusion system 600 and interface between aspects of the infusion system 600 and external devices and/or networks, for example, patient monitoring networks, instructional systems (e.g., an electronic medical record system), pharmacy systems, and/or nurse call systems, or as an interface to external equipment such as barcode readers to provide a means of inputting drug and/or patient information from medication or patient records. In some aspects, the infusion pump controller 660 is configured to provide physical attachment of the infusion system 600 to structures, such as intravenous pole 640 and/or bedrails, or the like.

### Example Syringe Pump

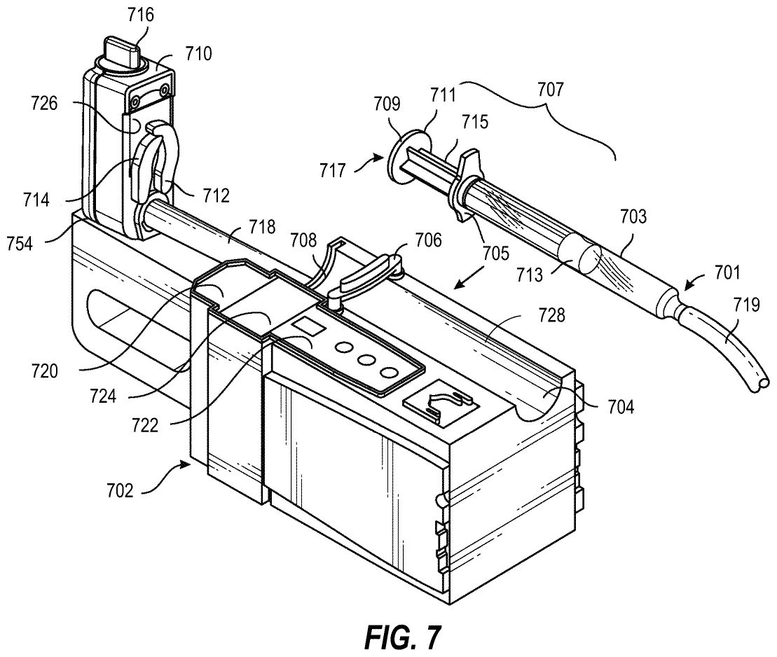

FIG. 7 depicts an example syringe pump 702. Syringe pump 702 includes a graphical user interface (GUI) 724 for displaying operational information, including the selected syringe type and the configured infusion (e.g., in terms of rate of infusion, volume infused, length of time of infusion, etc.). A user may operate syringe pump 702 through one or more buttons 722 in this example. Note that other examples may include other arrangements, such as touch-screen interfaces, or interfaces operable by remote device, such as by an application on a computing device.

Syringe 701 is shown next to syringe pump 702, rather than loaded in the pump, for clarity of illustration. Syringe pump 702 includes cradle 704 in which a barrel 703 of syringe 701 rests when mounted in the syringe pump 702. Sensor 728 detects volume markings along a barrel of syringe 701. Cradle 704 has a clamp 706 to securely hold the barrel 703 in a fixed position in cradle 704 to resist axial and lateral movement. Clamp 706 may pivot between an open position to permit loading or removal of syringe 701 and a closed position over cradle 704. Barrel clamp 706 may measure an outside diameter of the barrel 703 of syringe 701. Barrel flange 705 of syringe 701 resides in a barrel flange groove 708 in syringe pump 702 to immobilize barrel 703 from axial movement during movement of plunger 707 within barrel 703 of syringe 701. Barrel flange groove may measure an outside diameter and a thickness of the barrel flange 705 of syringe 701.

Plunger 707 can include push-button 709 having an inner side 711 and being interconnected with stopper 713 of plunger 707 by piston 715. When mounted in syringe pump 702, push-button 709 can be held by drive head 710 with a plunger retainer comprising a pair of pivotally mounted claws, first retainer claw 712 and second retainer claw 714, shown in the closed position in FIG. 7. The retainer claws 712 and 714 can curve inwardly toward each other to grasp push-button 709 mounted in syringe pump 702.

A rotation knob 716 can be used to control the positions of the first and second retainer claws 712 and 714 to allow removal and insertion of the push-button 709 and to release the split-nut from the driveshaft to permit axial positioning of the drive head 710. Syringes can be provided for use with a syringe pump with different quantities of fluid, and the plunger can be located at different positions in relation to the barrel.

The drive head 710 can allow manual adjustment to accommodate syringes with different beginning plunger positions. A syringe inserted in the cradle 704 can align with the drive head 710 within a particular axial range. The points where the axial center lines of the syringe intersect the driver can change according to the size of the syringe but only in one direction along the drive head 710. A guide device 718 can extend from the drive head 710 to a point within a body of the syringe pump 702.

Syringe pump 702 can include a control panel 720 providing multiple buttons 722 for control of the syringe pump 702 as well as GUI 724 used to present pump-specific information to the operator. The buttons 722 can allow the operator to program the syringe pump 702 for the flow rate, the volume to be infused, and other pump parameters. GUI 724 can present the programmed flow rate, the amount of fluid remaining to be infused, as well as alarms and other information.

The drive head 710 can include a contact plate 726 that has a pushing surface that contacts the outer side 717 of the push-button 709 as the drive head 710 moves forward toward the barrel 703, pushing the plunger 707 into the barrel 703 of the syringe to expel the syringe contents through a fluid administration set tubing 719 to a patient. A drive mechanism 754 is configured to drive the drive head 710 forward. The drive mechanism 754 may include, a drivetrain, for example, gears, axles, shafts, chains, hydraulics, and/or any other components for translating rotational motion of a motor into linear motion of the drive head 710. The drivetrain may include linear actuating components, such as a linear stepper motor, a brushed DC electric motor, a brushless DC electric motor, a servo motor, an AC motor, or any other type of motor.

When the contact plate 726 exerts force against the push-button 709, the force may be detected by a force sensor and transmitted to a processor for monitoring. In the case of friction between the stopper 713 and the barrel 703, the force exerted can increase and can be detected by a force sensor.

Syringe pump 702 is configured with known syringe types containing information such as syringe inner diameter and stroke of the stopper 713. Syringe pump 702 determines the position of stopper 713 based on the movement of drive head 710 based on the syringe type and stored characteristics of the syringe type. Then, syringe pump 702 calculates the volume infused, time elapsed, volume remaining, and time remaining. As the drive head 710 continues to move, a flow rate is determined based on the characteristics of the syringe and the velocity of the drive head 710.

One or more sensors, such as sensor 728, barrel clamp 706, groove 708, claws 712 and 714, and a force sensor, may measure various characteristics of syringe 701, to obtain a measured value for the characteristic, such as barrel length, barrel outside diameter, flange shape, flange size, plunger shape, plunger size (e.g., diameter), plunger tab thickness, volume per plunger distance moved, driver head height, etc. These sensors may include one or more of an optical sensor, a light source, a pressure sensor, a position sensor (e.g., magnetic linear position sensor indicating the plunger head), force sensor, or the like.

### Example Method for Contacting a Nurse using an Infusion Pump System

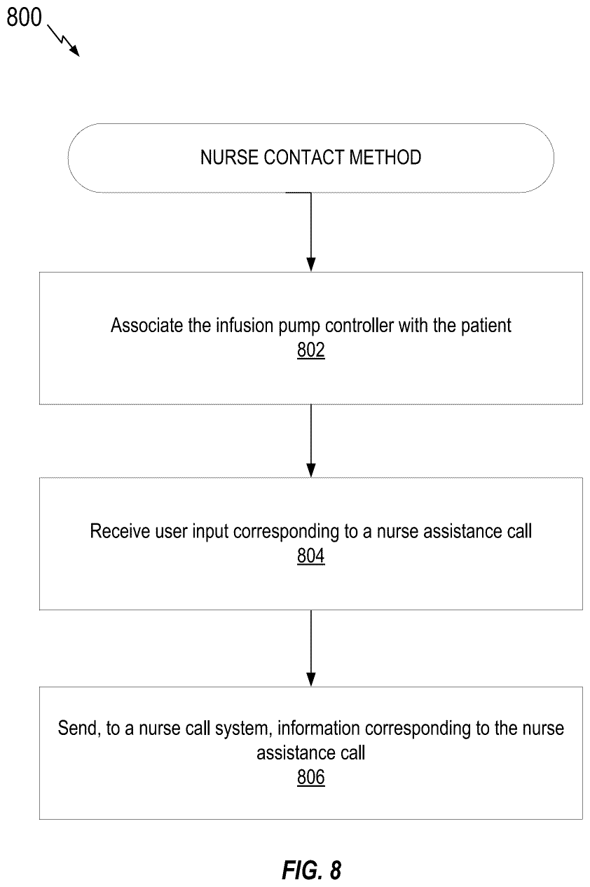

FIG. 8 shows an example method 800 for contacting a nurse using an infusion pump system, the infusion pump system comprising an infusion pump coupled to an infusion pump controller configured to control the infusion pump to deliver fluid to a patient, such as infusion system 600 or syringe pump 702.

Method 800 begins at step 802 with associating the infusion pump controller with the patient.

Method 800 then proceeds to step 804 with receiving, on the infusion pump controller, user input corresponding to a nurse assistance call.

Method 800 then proceeds to step 806 with sending, to a nurse call system, information corresponding to the nurse assistance call.

In some aspects, method 800 further includes outputting, at the infusion pump controller, a response to the nurse assistance call.

In some aspects, outputting the response comprises displaying the response.

In some aspects, method 800 further includes receiving, from the nurse call system, the response to the nurse assistance call.

In some aspects, method 800 further includes generating, by a language generation model, the response to the nurse assistance call based on the information.

In some aspects, outputting the response comprises outputting audio corresponding to the response.

In some aspects, the response comprises an acknowledgment of receipt of the nurse assistance call.

In some aspects, the response comprises a self-help message responsive to the nurse assistance call.

In some aspects, method 800 further includes determining a priority level of the nurse assistance call, wherein the priority level is based on sentiment analysis of the user input, and wherein the information includes the priority level.

In some aspects, method 800 further includes determining an action for the infusion pump controller based on the nurse assistance call; and controlling, the infusion pump controller based on the action.

In some aspects, the user input comprises free-form text input.

In some aspects, the user input comprises audio input; and the method further comprises activating a microphone coupled to the infusion pump controller; and capturing, with the microphone, the audio input.

In some aspects, method 800 further includes processing the audio input with a speech recognition model to generate a text transcript, wherein the information comprises the text transcript.

In some aspects, sending, to the nurse call system, the information corresponding to the nurse assistance call comprises initiating a telephone call between the infusion pump controller and the nurse call system.

In some aspects, method 800 further includes displaying, on the infusion pump controller, one or more preset nurse call requests, wherein the user input comprises selection of at least one of the one or more preset nurse call requests.

In some aspects, method 800 further includes, prior to receipt of the user input receiving, on the infusion pump controller, a second user input indicating to initiate a nurse call mode of the infusion pump controller; and displaying, in the nurse call mode, on the infusion pump controller, one or more user interface elements, wherein the one or more user interface elements are configured to receive the user input; or the one or more user interface elements are associated with one or more buttons configured to receive the user input.

In some aspects, method 800 further includes determining the infusion pump controller is in a locked mode determining the infusion pump is not associated with an active alarm; and initiating a nurse call mode of the infusion pump controller.

In some aspects, method 800 further includes receiving, on the infusion pump controller, a second user input indicating to initiate a nurse call mode of the infusion pump controller identifying, based on at least one of: a location of the infusion pump, a patient identifier of the patient, or a clinician identifier of a clinician, a preset nurse call user interface element; and displaying, in the nurse call mode, on the infusion pump controller, one or more user interface elements including the preset nurse call user interface element.

In some aspects, associating the infusion pump controller with the patient comprises receiving a patient identifier of the patient; and associating the patient identifier of the patient with the infusion pump controller.

In some aspects, method 800 further includes determining the infusion pump controller is not associated with the patient disabling receipt of the user input corresponding to the nurse assistance call receiving a patient identifier of the patient to associate with the infusion pump controller; and enabling receipt of the user input corresponding to the nurse assistance call.

In some aspects, method 800 further includes presenting, on a touchscreen display associated with the infusion pump controller, graphical control elements; and receiving, via the touchscreen display associated with the infusion pump controller, user inputs including the user input.

In some aspects, the information further comprises at least one of: a patient identifier of the patient, a time stamp of receipt of the user input, an identifier associated with the infusion pump controller, or an identifier of location of the infusion pump controller.

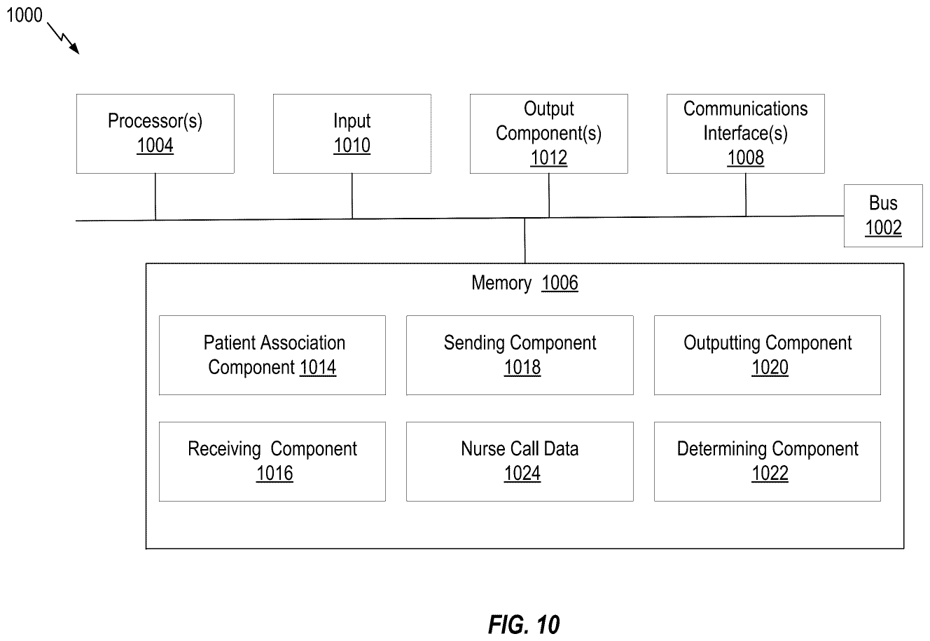

In one aspect, method 800, or any aspect related to it, may be performed by an apparatus, such as computing device 1000 of FIG. 10, which includes various components operable, configured, or adapted to perform the method 800. Computing device 1000 is described below in further detail.

Note that FIG. 8 is just one example of a method, and other methods including fewer, additional, or alternative steps are possible consistent with this disclosure.

### Example Method for Contacting a Nurse using an Infusion Pump System

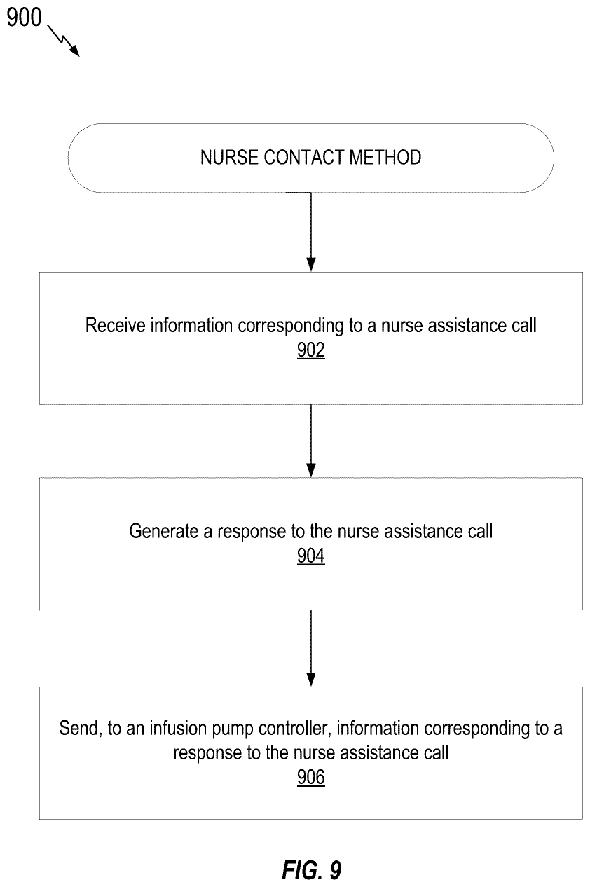

FIG. 9 shows an example method 900 for facilitating a nurse call, for example, as a nurse component 106 or nurse alert component 108.

Method 900 begins at step 902 with receiving, from an infusion pump controller, information corresponding to a nurse assistance call.

Method 900 proceeds to step 904 with generating a response to the nurse assistance call.

Method 900 then proceeds to step 906 with sending, to the infusion pump controller, information corresponding to a response to the nurse assistance call.

In some aspects, method 900 further includes sending, to a nurse call system, the information corresponding to the nurse assistance call; and receiving, from the nurse call system, a nurse response to the nurse assistance call, wherein the response to the nurse assistance call comprises the nurse response.

In some aspects, generating the response to the nurse assistance call, comprises processing the information corresponding to the nurse assistance call with a language generation model to generate the response.

In some aspects, method 900 further includes determining a priority level of the nurse assistance call, wherein the priority level is based on sentiment analysis of the information corresponding to a response to the nurse assistance call, and wherein the information includes the priority level.

In some aspects, the response comprises an acknowledgment of receipt of the nurse assistance call.

In some aspects, the response comprises a self-help message responsive to the nurse assistance call.

In some aspects, generating the response to the nurse assistance call, comprises receiving, from the nurse call system, a nurse response to the information, wherein the response comprises the nurse response responsive to the nurse assistance call.

In some aspects, method 900 further includes determining an action for the infusion pump controller based on the nurse assistance call, wherein the response indicates the action.

In some aspects, the information corresponding to the nurse assistance call comprises audio data, and the method further comprises processing the audio data with a speech recognition model to generate a text transcript, wherein the response to the nurse assistance call is generated based on the text transcript.

In some aspects, the information corresponding to the nurse assistance call comprises a preset nurse call, and the method further comprises selecting, from a set of preset responses, a preset response based on the preset nurse call, wherein the information corresponding to the response to the nurse assistance call comprises the preset response.

In some aspects, the information further comprises at least one of: a patient identifier of the patient, a time stamp of receipt of the nurse assistance call input, an identifier associated with the infusion pump controller, or an identifier of a location of the infusion pump controller.

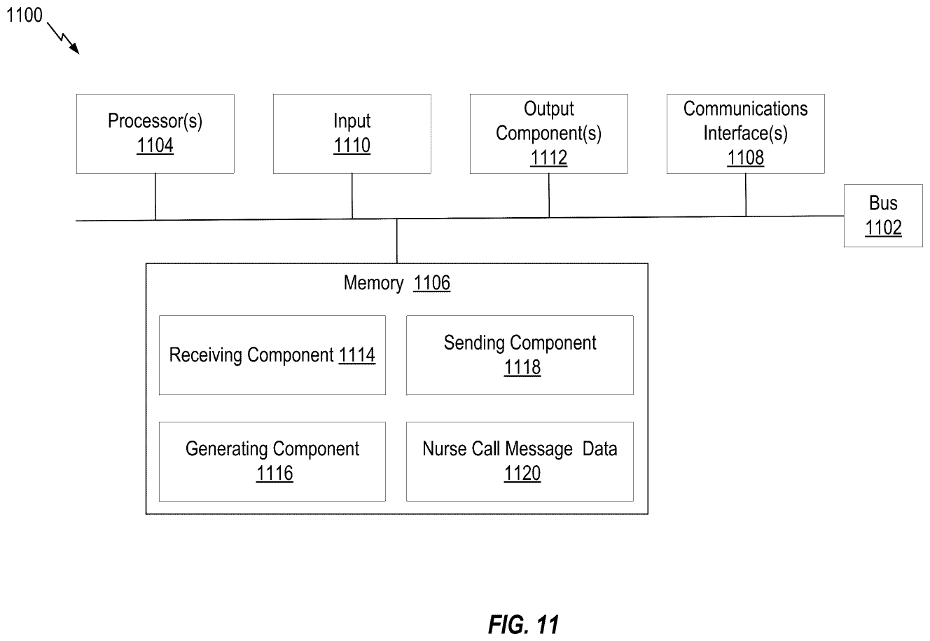

In one aspect, method 900, or any aspect related to it, may be performed by an apparatus, such as computing device 1100 of FIG. 11, which includes various components operable, configured, or adapted to perform the method 900. Computing device 1100 is described below in further detail.

Note that FIG. 9 is just one example of a method, and other methods including fewer, additional, or alternative steps are possible consistent with this disclosure.

### Example Computing Device for Contacting a Nurse using an Infusion Pump System

FIG. 10 depicts an example computing device 1000, such as a medical device, which implements various features and processes described herein, such as infusion system 600 in FIG. 6 and/or syringe pump 702. For example, the computing device 1000 may perform one or more steps of any of workflow 200 or workflow 300 or method 800. The computing device 1000 may include one or more processors 1004, one or more memories 1006, one or more input components 1010, one or more output components 1012, and one or more communication interfaces 1008. Each of these components may be coupled by a bus 1002.

Computing device 1000 may perform these processes based on one or more processors 1004 executing software instructions stored by a computer-readable medium, such as one or more memories 1006. In certain embodiments, one or more processors 1004 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 1008. When executed, software instructions stored in one or more memories may cause one or more processors 1004 to perform one or more processes described herein.

A memory 1006 may include data storage or one or more data structures (e.g., a database, etc.). Computing device 1000 may be capable of receiving information from, storing information in, communicating information to, or searching information stored in the data storage or one or more data structures in one or more memories 1006.

A memory 1006 may include random access memory (RAM), read only memory (ROM), and/or other types of dynamic or static storage devices (e.g., flash memory, magnetic memory, optical memory, etc.), that stores information and/or instructions for use by one or more processors 1004. For example, a memory 1006 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, one or more memories 1006 may include a patient association component 1014 is configured for associating the infusion pump controller with the patient.

In some aspects, one or more memories 1006 may include receiving component 1016 is configured for receiving, on the infusion pump controller, user input corresponding to a nurse assistance call; or receiving, from the nurse call system, the response to the nurse assistance call.

In some aspects, one or more memories 1006 may include sending component 1018 is configured for sending, to a nurse call system, information corresponding to the nurse assistance call.

In some aspects, one or more memories 1006 may include outputting component 1020 is configured for outputting, at the infusion pump controller, a response to the nurse assistance call; outputting audio corresponding to the response.

In some aspects, one or more memories 1006 may include determining component 1022 is configured for includes determining a priority level of the nurse assistance call, wherein the priority level is based on sentiment analysis of the user input, and wherein the information includes the priority level; generating, by a language generation model, the response to the nurse assistance call based on the information; or determining the infusion pump controller is in a locked mode determining the infusion pump is not associated with an active alarm; and initiating a nurse call mode of the infusion pump controller.

In some aspects, one or more memories 1006 may include nurse call data 1024 used by receiving component 1016 and/or sending component 1018.

One or more processors 1004 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 1010 may include a component that permits computing device 1000 to receive information, such as via user input (e.g., a touch-screen display, a keyboard, a keypad, a mouse, a button, a switch, a microphone, etc.). Further, one or more input components 1010 may include a sensor for sensing information (e.g., a global positioning system (GPS) component, an accelerometer, a gyroscope, an actuator, etc.).

One or more output components 1012 may include a component that provides output information from computing device 1000 (e.g., a display, a speaker, one or more light-emitting diodes (LEDs), etc.).

Communication interface 1008 may include a transceiver-like component (e.g., a transceiver, a separate receiver and transmitter, etc.) that enables computing device 1000 to communicate with other devices, such as via a wired connection, a wireless connection, or a combination of wired and wireless connections. Communication interface 1008 may permit computing device 1000 to receive information from another device and/or provide information to another device. For example, communication interface 1008 may include an Ethernet interface, an optical interface, a coaxial interface, an infrared interface, a radio frequency (RF) interface, a universal serial bus (USB) interface, a Wi-Fi interface, a cellular network interface, and/or the like.

### Example Computing Device for Contacting a Nurse using an Infusion Pump System

FIG. 11 depicts another example computing device 1100, such as a medical device, which implements various features and processes described herein. For example, the computing device 1100 may perform one or more steps of any of workflow 300 or method 900. The computing device 1100 may include one or more processors 1104, one or more memories 1106, one or more input components 1110, one or more output components 1112, and one or more communication interfaces 1108. Each of these components may be coupled by a bus 1102.

Computing device 1100 may perform these processes based on one or more processors 1104 executing software instructions stored by a computer-readable medium, such as one or more memories 1106. In certain embodiments, one or more processors 1104 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 1108. When executed, software instructions stored in one or more memories may cause one or more processors 1104 to perform one or more processes described herein.

A memory 1106 may include data storage or one or more data structures (e.g., a database, etc.). Computing device 1100 may be capable of receiving information from, storing information in, communicating information to, or searching information stored in the data storage or one or more data structures in one or more memories 1106.

A memory 1106 may include random access memory (RAM), read only memory (ROM), and/or other types of dynamic or static storage devices (e.g., flash memory, magnetic memory, optical memory, etc.), that stores information and/or instructions for use by one or more processors 1104. For example, a memory 1106 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, one or more memories 1106 may include receiving component 1114 is configured for receiving, from an infusion pump controller, information corresponding to a nurse assistance call.

In some aspects, one or more memories 1106 may include generating component 1116 is configured for generating a response to the nurse assistance call.

In some aspects, one or more memories 1106 may include sending component 1118 is configured for sending, to the infusion pump controller, information corresponding to a response to the nurse assistance call.

In some aspects, one or more memories 1106 may include nurse call message data 1120 used by generating component 1116.

One or more processors 1104 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 1110 may include a component that permits computing device 1100 to receive information, such as via user input (e.g., a touch-screen display, a keyboard, a keypad, a mouse, a button, a switch, a microphone, etc.). Further, one or more input components 1110 may include a sensor for sensing information (e.g., a global positioning system (GPS) component, an accelerometer, a gyroscope, an actuator, etc.).

One or more output components 1112 may include a component that provides output information from computing device 1100 (e.g., a display, a speaker, one or more light-emitting diodes (LEDs), etc.).

Communication interface 1108 may include a transceiver-like component (e.g., a transceiver, a separate receiver and transmitter, etc.) that enables computing device 1100 to communicate with other devices, such as via a wired connection, a wireless connection, or a combination of wired and wireless connections. Communication interface 1108 may permit computing device 1100 to receive information from another device and/or provide information to another device. For example, communication interface 1108 may include an Ethernet interface, an optical interface, a coaxial interface, an infrared interface, a radio frequency (RF) interface, a universal serial bus (USB) interface, a Wi-Fi interface, a cellular network interface, and/or the like.

### EXAMPLE CLAUSES

Implementation examples are described in the following numbered clauses:

Clause 1: A method of contacting a nurse using an infusion pump system, the infusion pump system comprising an infusion pump coupled to an infusion pump controller configured to control the infusion pump to deliver fluid to a patient, the method comprising associating the infusion pump controller with the patient; receiving, on the infusion pump controller, user input corresponding to a nurse assistance call; and sending, to a nurse call system, information corresponding to the nurse assistance call, the information identifying at least one of the infusion pump controller or the patient.

Clause 2: The method of clause 1, further comprising outputting, at the infusion pump controller, a response to the nurse assistance call.

Clause 3: The method of clause 2, wherein outputting the response comprises displaying the response.

Clause 4: The method of clause 3, further comprising receiving, from the nurse call system, the response to the nurse assistance call.

Clause 5: The method of any one of clauses 3-4, further comprising generating, by a language generation model, the response to the nurse assistance call based on the information.

Clause 6: The method of any one of clauses 2-5, wherein outputting the response comprises outputting audio corresponding to the response.

Clause 7: The method of any one of clauses 2-6, wherein the response comprises an acknowledgment of receipt of the nurse assistance call.

Clause 8: The method of any one of clauses 2-7, wherein the response comprises a self-help message responsive to the nurse assistance call.

Clause 9: The method of any one of clauses 1-8, further comprising determining a priority level of the nurse assistance call, wherein the priority level is based on sentiment analysis of the user input, and wherein the information includes the priority level.

Clause 10: The method of any one of clauses 1-9, further comprising determining an action for the infusion pump controller based on the nurse assistance call; and controlling, the infusion pump controller based on the action.

Clause 11: The method of any one of clauses 1-10, wherein the user input comprises free-form text input, and wherein the information corresponding to the nurse assistance call includes the free-form text input.

Clause 12: The method of any one of clauses 1-11, wherein the user input comprises audio input; and the method further comprises activating a microphone coupled to the infusion pump controller; and capturing, with the microphone, the audio input.

Clause 13: The method of clause 12, further comprising processing the audio input with a speech recognition model to generate a text transcript, wherein the information corresponding to the nurse assistance call includes the text transcript.

Clause 14: The method of any one of clauses 1-13, wherein sending, to the nurse call system, the information corresponding to the nurse assistance call comprises initiating a telephone call between the infusion pump controller and the nurse call system.

Clause 15: The method of any one of clauses 1-14, further comprising displaying, on the infusion pump controller, one or more preset nurse call requests, wherein the user input comprises selection of at least one of the one or more preset nurse call requests.

Clause 16: The method of any one of clauses 1-15, further comprising, prior to receipt of the user input receiving, on the infusion pump controller, a second user input indicating to initiate a nurse call mode of the infusion pump controller; and displaying, in the nurse call mode, on the infusion pump controller, one or more user interface elements, wherein the one or more user interface elements are configured to receive the user input; or the one or more user interface elements are associated with one or more buttons configured to receive the user input.

Clause 17: The method of any one of clauses 1-16, further comprising determining the infusion pump controller is in a locked mode determining the infusion pump is not associated with an active alarm; and initiating a nurse call mode of the infusion pump controller.

Clause 18: The method of any one of clauses 1-17, further comprising receiving, on the infusion pump controller, a second user input indicating to initiate a nurse call mode of the infusion pump controller identifying, based on at least one of: an identifier of a location of the infusion pump, a patient identifier of the patient, or a clinician identifier of a clinician, a preset nurse call user interface element; and displaying, in the nurse call mode, on the infusion pump controller, one or more user interface elements including the preset nurse call user interface element.

Clause 19: The method of any one of clauses 1-18, wherein associating the infusion pump controller with the patient comprises receiving a patient identifier of the patient; and associating the patient identifier of the patient with the infusion pump controller.

Clause 20: The method of any one of clauses 1-19, wherein associating the infusion pump controller with the patient comprises receiving a location identifier of at least one of the patient or the infusion pump controller, wherein the information corresponding to the nurse assistance call includes the location identifier.

Clause 21: The method of any one of clauses 1-20, further comprising determining the infusion pump controller is not associated with the patient disabling receipt of the user input corresponding to the nurse assistance call receiving a patient identifier of the patient to associate with the infusion pump controller; and enabling receipt of the user input corresponding to the nurse assistance call.

Clause 22: The method of any one of clauses 1-21, further comprising presenting, on a touchscreen display associated with the infusion pump controller, graphical control elements; and receiving, via the touchscreen display associated with the infusion pump controller, user inputs including the user input associated with one or more of the graphical control elements.

Clause 23: The method of any one of clauses 1-22, wherein the information further comprises at least one of: a patient identifier of the patient, a time stamp of receipt of the user input, an identifier associated with the infusion pump controller, or an identifier of a location of the infusion pump controller.

Clause 24: A method of facilitating a nurse call, comprising receiving, from an infusion pump controller, information corresponding to a nurse assistance call, the information identifying at least one of a patient or the infusion pump controller; generating a response to the nurse assistance call; and sending, to the infusion pump controller, information corresponding to a response to the nurse assistance call.

Clause 25: The method of clause 24, further comprising sending, to a nurse call system, the information corresponding to the nurse assistance call; and receiving, from the nurse call system, a nurse response to the nurse assistance call, wherein the response to the nurse assistance call comprises the nurse response.

Clause 26: The method of any one of clauses 24-25, wherein generating the response to the nurse assistance call, comprises processing the information corresponding to the nurse assistance call with a language generation model to generate the response.

Clause 27: The method of any one of clauses 24-26, further comprising determining a priority level of the nurse assistance call, wherein the priority level is based on sentiment analysis of the information corresponding to a response to the nurse assistance call, and wherein the information includes the priority level.

Clause 28: The method of any one of clauses 24-27, wherein the response comprises an acknowledgment of receipt of the nurse assistance call.

Clause 29: The method of any one of clauses 24-28, wherein the response comprises a self-help message responsive to the nurse assistance call.

Clause 30: The method of any one of clauses 24-29, wherein generating the response to the nurse assistance call, comprises receiving, from the nurse call system, a nurse response to the information, wherein the response comprises the nurse response responsive to the nurse assistance call.

Clause 31: The method of any one of clauses 24-30, further comprising determining an action for the infusion pump controller based on the nurse assistance call, wherein the response indicates the action.

Clause 32: The method of any one of clauses 24-31, wherein the information corresponding to the nurse assistance call comprises audio data, and the method further comprises processing the audio data with a speech recognition model to generate a text transcript, wherein the response to the nurse assistance call is generated based on the text transcript.

Clause 33: The method of any one of clauses 24-32, wherein the information corresponding to the nurse assistance call comprises a preset nurse call, and the method further comprises selecting, from a set of preset responses, a preset response based on the preset nurse call, wherein the information corresponding to the response to the nurse assistance call comprises the preset response.

Clause 34: The method of any one of clauses 24-33, wherein the information further comprises at least one of: a patient identifier of the patient, a time stamp of receipt of the nurse assistance call input, an identifier associated with the infusion pump controller, or an identifier of a location of the infusion pump controller.

Clause 35: An infusion system, comprising: an infusion pump configured to deliver fluid to a patient; and an infusion pump controller configured to perform a method in accordance with any one of Clauses 1-23.

Clause 36: A processing system, comprising: memory comprising computer-executable instructions; and one or more processors configured to execute the computer-executable instructions and cause the processing system to perform a method in accordance with any one of Clauses 1-34.

Clause 37: A processing system, comprising means for performing a method in accordance with any one of Clauses 1-34.

Clause 38: A non-transitory computer-readable medium storing program code for causing a processing system to perform the steps of any one of Clauses 1-34.

Clause 39: A computer program product embodied on a computer-readable storage medium comprising code for performing a method in accordance with any one of Clauses 1-34.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database, or another data structure), ascertaining or the like. Also, “determining” may include receiving (e.g., receiving information), accessing (e.g., accessing data in a memory) or the like. Also, “determining” may include resolving, selecting, choosing, establishing or the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of contacting a nurse using an infusion pump system, the infusion pump system comprising an infusion pump coupled to an infusion pump controller configured to control the infusion pump to deliver fluid to a patient, the method comprising:
associating the infusion pump controller with the patient;
receiving, on the infusion pump controller, user input corresponding to a nurse assistance call; and
sending, to a nurse call system, information corresponding to the nurse assistance call, the information identifying at least one of the infusion pump controller or the patient.

2. The method of claim 1, further comprising outputting, at the infusion pump controller, a response to the nurse assistance call.

3. The method of claim 2, wherein outputting the response comprises displaying the response.

4. The method of claim 3, further comprising receiving, from the nurse call system, the response to the nurse assistance call.

5. The method of claim 2, further comprising generating, by a language generation model, the response to the nurse assistance call based on the information; wherein the response comprises a self-help message responsive to the nurse assistance call.

6. The method of claim 2, wherein the response comprises an acknowledgment of receipt of the nurse assistance call.

7. The method of claim 1, further comprising determining a priority level of the nurse assistance call, wherein the priority level is based on sentiment analysis of the user input, and wherein the information includes the priority level.

8. The method of claim 1, further comprising:
determining an action for the infusion pump controller based on the nurse assistance call; and
controlling, the infusion pump controller based on the action.

9. The method of claim 1, wherein the user input comprises free-form text input, and wherein the information corresponding to the nurse assistance call includes the free-form text input.

10. The method of claim 1, wherein the user input comprises audio input; and the method further comprises:
activating a microphone coupled to the infusion pump controller;
capturing, with the microphone, the audio input; and processing the audio input with a speech recognition model to generate a text transcript, wherein the information corresponding to the nurse assistance call includes the text transcript.

11. The method of claim 1, wherein sending, to the nurse call system, the information corresponding to the nurse assistance call comprises initiating a telephone call between the infusion pump controller and the nurse call system.

12. The method of claim 1, further comprising displaying, on the infusion pump controller, one or more preset nurse call requests, wherein the user input comprises selection of at least one of the one or more preset nurse call requests.

13. The method of claim 1, further comprising:
determining the infusion pump controller is in a locked mode;
determining the infusion pump is not associated with an active alarm; and
initiating a nurse call mode of the infusion pump controller.

14. The method of claim 1, wherein the information further comprises at least one of: a patient identifier of the patient, a time stamp of receipt of the user input, an identifier associated with the infusion pump controller, or an identifier of a location of the infusion pump controller.

15. A method of facilitating a nurse call, comprising:
receiving, from an infusion pump controller, information corresponding to a nurse assistance call, the information identifying at least one of a patient or the infusion pump controller;
generating a response to the nurse assistance call; and
sending, to the infusion pump controller, information corresponding to a response to the nurse assistance call.

16. The method of claim 15, further comprising:
sending, to a nurse call system, the information corresponding to the nurse assistance call; and
receiving, from the nurse call system, a nurse response to the nurse assistance call, wherein the response to the nurse assistance call comprises the nurse response.

17. The method of claim 15, wherein generating the response to the nurse assistance call, comprises processing the information corresponding to the nurse assistance call with a language generation model to generate the response.

18. The method of claim 15, further comprising determining a priority level of the nurse assistance call, wherein the priority level is based on sentiment analysis of the information corresponding to a response to the nurse assistance call, and wherein the information includes the priority level.

19. The method of claim 15, wherein the response comprises at least one of an acknowledgment of receipt of the nurse assistance call or a self-help message responsive to the nurse assistance call.

20. The method of claim 15, further comprising:
determining an action for the infusion pump controller based on the nurse assistance call, wherein the response indicates the action.
